(19) United States
(12) Patent Application Publication
(10) Pub. No.: US 2014/0186821 A1
(43) Pub. Date: Jul. 3, 2014

(54) Noninterfering Multipurpose Compositions for Collecting, Transporting and Storing Biological Samples

(71) Applicant: Longhorn Vaccines and Diagnostics, LLC, Bethesda, MD (US)

(72) Inventors: Luke T. Daum, San Antonio, TX (US); Gerald W. Fischer, Bethesda, MD (US)

(73) Assignee: Longhorn Vaccines and Diagnostics, LLC, Bethesda, MD (US)

(21) Appl. No.: 14/136,758

(22) Filed: Dec. 20, 2013

Related U.S. Application Data
(60) Provisional application No. 61/746,962, filed on Dec. 28, 2012.

(51) Int. Cl. C12Q 1/68

(52) U.S. Cl. CPC C12Q 1/6806
USPC 435/5; 435/6.12; 435/309.1

(57) ABSTRACT

The invention is directed to compositions and methods for collecting, transporting, and storing microorganisms obtained from samples of biological, clinical, forensic, and environmental origin. Compositions preserve the viability of the collected organisms, permit long-term storage, and are compatible with subsequent manipulation including propagation and culture of collected microorganisms, or isolation, purification, detection, and characterization of proteins, nucleic acids and macromolecules. When the compositions containing microorganisms and polynucleotides therein are further processed, such as by nucleic acid testing, there is an increased ability to detect, isolate, purify and/or characterize select microbes and their components, when compared to conventional microbial transport media that contain interfering substance(s). In particular formulations, the compositions allow biological samples to be collected, transported, and even stored for extended periods, and are compatible with nucleic acid extraction, identification, quantitation, PCR amplification, and genomic analysis methodologies.

### REFERENCE TO RELATED APPLICATIONS

This Application claims priority to U.S. Provisional Application No. 61/746,962 of the same title, filed Dec. 28, 2012, the entirety of which is hereby specifically incorporated by reference.

### BACKGROUND

1. Field of the Invention

The present invention provides compositions and methods for nucleic acid detection and analysis from collection to analysis with mixtures, solutions and media that contain gelatin whereby the media compositions of the invention are compatible with molecular analysis and the gelatin does not inhibit or impede nucleic acid extraction or analysis such as detection by polymerase chain reaction procedures.

2. Description of the Background

Before the advent of molecular techniques, the majority of clinical diagnostic laboratories employed the sole use of traditional culturing methods that typically require three to seven days for a viral culture—and even longer for bacterial targets. Although recent advances in cell culture methods have resulted in quicker culturing times, these cell culturing and propagation techniques are used mainly for confirmatory diagnostic purposes and are still viewed as the standard by which other methods are compared. Differing from molecular methods, cell culture techniques require the maintenance of viability of the organism present in a collected sample. Even analysis of cellular components such as blood cells and tissue biopsies often required viable or intact cells. Currently, most laboratories combine various culture and non-culture techniques to optimize analysis of microbes or host cells of a particular pathogen.

Conventional collection and transport media (e.g., viral transport media, microbial or bacterial transport media, parasite transport media, fungal transport media, environmental sample transport media) have traditionally been developed based on cell culture-related requirements or growth requirements of the collected cells or organism(s), rather than for the purpose of molecular techniques, such as isolating or preserving nucleic acids from the sample for subsequent nucleic acid analysis. For example, the Centers for Disease Control and Prevention (CDC) require that the collection of respiratory viral samples (including nasal washes, throat swabs and nasopharyngeal swabs, and other biological samples), be performed in collection media that were originally developed solely to maintain the viability of collected specimens until they were cultured in the laboratory. These molecular transport media were not formulated with the consideration that, in addition to traditional viral propagation and cell culture methodologies, a large portion of microbial identification and analysis done today employs molecular assays (commonly referred to as “nucleic acid testing,” or “NAT”). Thus, most commercially-available transport culture media (including, for example, Remel's MicroTest™ M4RT®, Copan's Universal Transport Medium (UTM-RT), Becton Dickinson's Universal Viral Transport Medium, and the like), were originally designed primarily for cell culture and viral propagation, and not necessarily formulated to be compatible with subsequent NAT procedures. Accordingly, there is a need in the art for mixtures, solutions and media that do not substantially interfere with downstream molecular analysis. Such solutions may be used for propagation of microorganisms or molecular assays, e.g., NAT.

### SUMMARY OF THE INVENTION

The present invention overcomes the problems and disadvantages associated with current strategies and designs and provides new tools, compositions and methods for collecting, transporting and storing biological samples preferably for later diagnostic analysis.

One embodiment of the invention is directed to a method of detecting a sequence in a biological sample comprising: contacting the biological sample at ambient temperature with an effective amount of an transport medium containing a stabilizing agent that functions to maintain the integrity of nucleic acid sequences contained within the biological sample without interfering with subsequent molecular analysis of the sample. Preferably the biological sample comprises cells suspected of being infected with a pathogen and the pathogen is a viral, a bacterial, a parasitic or a fungal infection. Also preferably the transport medium is a collection, transport and storage medium and provided in at least a volume equivalent to the volume of the biological sample, and more preferably at least three times the volume of the biological sample. Preferably the stabilizing agent is one or more of a serum albumin, agar, carrageenan, gelatin, pectin, a sugar polymer, a galactose polymer, a polysaccharide, a heteropolysaccharide, a linear-sulfated polysaccharide, a protein, a collagen or a hydrolyzed product of collagen, that does not inhibit subsequent molecular analysis and the preferred molecular analysis is PCR. More preferably, the stabilizing agent is a gelatin obtained or derived from fish or fish products. Preferably, the polymerase chain reaction cycle threshold (C_{T}) value that equates to about 10 to 100-fold lower than that obtained when the biological sample is maintain in a medium containing bovine serum albumin, and further, 80 percent or more of the nucleic acid sequences present in the biological sample are detectable for at least 30 days.

Another embodiment of the invention is directed to a method of culturing a predetermined microorganism from a biological sample comprising: contacting the biological sample suspected of containing the microorganism with an effective of an aqueous collection, transport or storage (CTS) medium to form a mixture, where the CTS medium contains from about 1 to 10 g/L of a buffer, from about 10 to 50 g/L of trehalose, from about 1 to 10 g/L of a carbohydrate comprising at least one of glucose, sucrose, mannose, altrose, allose, idose, talose, fructose, methyl-α-D-glucoside, galactose, ribose, deoxyribose, xylose, lactose, maltose, glycogen, amylase, cellulose, 6-deoxy-α-d-gluco-heptopyranosyl 6-deoxy-α-d-gluco-heptopyranoside, or (6-deoxy-α-d-gluco-heptopyranosyluronic acid) 6-deoxy-α-d-gluco-heptopyranosiduronic acid, or any combination thereof; and up to about 2 g/L of a serum albumin that does not interfere with quantitative detection of nucleic acid sequences by subsequent molecular analysis procedure; inoculating a growth medium suitable for the microorganism with an effective amount of the mixture; and culturing the mixture under conditions and for a period of time sufficient to obtain growth of the microorganism. Preferably the aqueous collection, transport and storage (CTS) medium comprises about 1 to about 10 g/L of a buffer, about 10 to about 50 g/L of trehalose; about 1 to about 10 g/L of a carbohydrate comprising glucose, sucrose, mannose, altrose, allose, idose, talose, fructose, methyl-α-D-glucoside, galactose, ribose, deoxyribose, xylose, lactose, maltose, glycogen, amylase, cellulose, 6-deoxy-α-d-gluco-heptopyranosyl 6-deoxy-α-d-gluco-heptopyranoside, or (6-deoxy-α-d-gluco-heptopyranosyluronic acid) 6-deoxy-α-d-gluco-heptopyranosiduronic acid, or any combination thereof; and up to about 2 g/L of a serum albumin, wherein the medium contains no substances at a concentration that interferes with the detection of nucleic acid as compared to a medium containing bovine gelatin. Also preferably, the interfering substance is at least one of a sugar polymer, a galactose polymer, a polysaccharide, a linear sulfated polysaccharide, a heteropolysaccharide, collagen or a product of hydrolyzed collagen, a protein, or any combination thereof. Preferably, the transport medium further comprises about 30 to about 40 g/L of trehalose, about 5 to about 10 g/L of each of fructose and glucose; and from about 1 to about 1.5 g/L of a serum albumin, wherein the buffer comprises N-tris(hydroxymethyl)methyl-2-aminoethanesulfonic acid (TES), 4-(2-hydroxyethyl)-1-piperazineethanesulfonic acid (HEPES), 4-(2-hydroxyethyl)piperazine-l-propanesulfonic acid (HEPPS), 2-morpholinoethanesulfonic acid monohydrate (MES), 3-morpholinopropanesulfonic acid (MOPS), piperazine-N,N′-bis(2-ethane sulfonic acid) (PIPES), N-tris(hydroxymethyl)methyl glycine (Tricine), or a combination thereof, and optionally at least one antibiotic which comprises vancomycin, polymyxin B, gentamycin, colistin, trimethoprim, amphotericin B, or a combination thereof, a salt which comprises calcium chloride, potassium chloride, magnesium chloride, sodium chloride, magnesium sulfate, or a combination thereof, a pH indicator which comprises neutral red, phenol red, or a combination thereof, and at least one amino acid which comprises glutamic acid, aspartic acid, or a combination thereof.

Another embodiment of the invention is directed to an aqueous collection, transport and storage (CTS) medium that comprises: about 0.001 mM to about 650 mM of trehalose, about 0.001 mM to about 600 mM of a carbohydrate comprising allose, altrose, arabinose, deoxyribose, erythrose, erythrulose, fructose, galactose, glucose, idose, lyxose, mannose, psicose, ribose, ribulose, sorbose, tagatose, talose, threose, xylose, xylulose, cellobiose, isomaltose, lactose, lactulose, maltose, maltulose, mannobiose, melibiose, sucrose, turanose, xylobiose, 6-deoxy-α-d-gluco-heptopyranosyluronic acid, 6-deoxy-α-d-gluco-heptopyranosiduronic acid, 6-deoxy-α-d-gluco-heptopyranosylic acid, 6-deoxy-α-d-gluco-heptopyranoside, cellulose, dextran, galactan, glycogen, levan, maltodextrin, maltotriose, mannan, melezitose, methyl-α-D-glucoside, raffinose, rhamnose, starch, or a combination thereof; about 1 μM to about 1 M of a buffer comprising BES, TES, HEPES, HEPPS, MES, MOPS, PIPES, Tricine, or a combination thereof; up to about 2 g/L of a serum albumin; about 1 μM to about 100 mM of a salt comprising calcium chloride, potassium chloride, magnesium chloride, sodium chloride, magnesium sulfate, or a combination thereof; about 0.1 μM to about 500 mM of an amino acid comprising glutamic acid, aspartic acid, or a combination thereof; about 1 μM to about 50 mM of a pH indicator comprising neutral red, phenol red, or a combination thereof; and about 0.1 μM to about 1 mM of an antibiotic comprising vancomycin, polymyoxin B, gentamycin, colistin, trimethoprim, amphotericin B, or a combination thereof.

Another embodiment of the invention is directed to an aqueous collection, transport and storage (CTS) medium that comprises: about 0.001 mM to about 650 mM of trehalose; about 0.001 mM to about 600 mM of at least a second carbohydrate comprising allose, altrose, arabinose, deoxyribose, erythrose, erythrulose, fructose, galactose, glucose, idose, lyxose, mannose, psicose, ribose, ribulose, sorbose, tagatose, talose, threose, xylose, xylulose, cellobiose, isomaltose, lactose, lactulose, maltose, maltulose, mannobiose, melibiose, sucrose, turanose, xylobiose, 6-deoxy-α-d-gluco-heptopyranosyluronic acid, 6-deoxy-α-d-gluco-heptopyranosiduronic acid, 6-deoxy-α-d-gluco-heptopyranosylic acid, 6-deoxy-α-d-gluco-heptopyranoside, cellulose, dextran, galactan, glycogen, levan, maltodextrin, maltotriose, mannan, melezitose, methyl-α-D-glucoside, raffinose, rhamnose, starch, and any combination thereof; about 0.001 mM to about 1000 mM of a buffer comprising BES, TES, HEPES, HEPPS, MES, MOPS, PIPES, Tricine, or any combination thereof; and from about 1 to about 35 μM of a serum albumin. Preferably, the medium further comprises about 1 μM to about 100 mM of a salt comprising calcium chloride, potassium chloride, magnesium chloride, sodium chloride, magnesium sulfate, or any combination thereof; about 0.1 μM to about 500 mM of an amino acid comprising glutamic acid, aspartic acid, or a combination thereof; about 1 μM to about 50 mM of a pH indicator comprising neutral red, phenol red, or a combination thereof; or about 0.1 μM to about 1 mM of an antibiotic comprising vancomycin, polymyoxin B, gentamycin, colistin, trimethoprim, amphotericin B, or any combination thereof, and has a pH of about 7.1 to about 7.5. Preferably, the aqueous medium of the invention is capable of sustaining the viability of a microorganisms in the biological sample, and contains no substances at concentrations that interfere with quantitative nucleic acid detection of a sequence of the microorganism by molecular analysis. Preferably, the biological sample containing the microorganisms is maintained in the CTS medium at a temperature of from about minus 80 C to about 37 C, for a period of from about one hour to about six months or, alternatively, the biological sample containing the microorganism is maintained in the CTS medium at a temperature of from about 0 C to about 30 C, for a period of from about one to about 90 days. Preferably the microorganism is Chlamydia, Mycoplasma, Ureaplasma, Adenovirus, Herpes Simplex Virus, Paramyxovirus, or

Influenza virus, wherein the Influenza virus is one or more of Influenza A, Influenza B, or Influenza C, or Influenza H1, H3, H5, or H1N1 subtype. Also preferably, the aqueous medium is compatible with isolation, detection, purification, and amplification of a nucleic acid sequence of the biological sample. Preferably amplification of the nucleic acid sequences is greater than or equal to about 1-log to the base 10 (about 3.3 C_{T}) when the sample is contacted by a conventional medium including at least one interfering substance.

Another embodiment of the invention is directed to a sample collection system that comprises: a sterile collection device adapted to obtain a biological sample suspected of containing a microorganism; and a sterile specimen container comprising the aqueous medium of claim 29 to which the biological sample is to be added. Preferably the microorganism is one or more of Chlamydia, Mycoplasma, Ureaplasma, Adenovirus, Herpes Simplex Virus, Paramyxovirus, or Influenza virus.

Another embodiment of the invention is directed to a method for amplifying a sequence from a nucleic acid contained in a biological sample comprising: contacting the biological sample with an effective amount of the aqueous medium of the invention to form a mixture; storing the mixture for a period of 30 days or more at a temperature of from about minus 10 C to about 35 C such that such at least 80 percent of the nucleic acid of the biological sample remain detectable; amplifying the sequence of the stored mixture; and detecting the amplified sequence. Preferably the period of time is greater than 3 months, detecting the amplified sequence is determined by measuring the polymerase chain reaction cycle threshold (C_{T}) of the sequence, and the polymerase chain reaction cycle threshold (C_{T}) value equates to about 10 to 100-fold lower than that obtained when a substantial amount of an interfering substance is present in the composition.

Other embodiments and advantages of the invention are set forth in part in the description, which follows, and in part, may be obvious from this description, or may be learned from the practice of the invention.

### DESCRIPTION OF THE DRAWINGS

FIG. 1A shows the cycle thresholds for extraction and RRT-PCT-inhibition of Influenza A (H3N2) virus from commercial VTMs.

FIG. 1B shows the cycle thresholds for extraction and RRT-PCT-inhibition of Mycobacterium tuberculosis (H37Rv) from commercial VTMs.

FIG. 1C shows the cycle thresholds for extraction and RRT-PCT-inhibition of Adenovirus 5 from commercial VTMs.

FIG. 2 shows the comparative cycle thresholds for the detection of Influenza A (whole virus) with water, PrimeStore and BD UTM.

FIG. 3 shows the cycle thresholds for the real-time RT-PCR detection of Influenza A (H3N2) RNA from gelatin solutions.

FIG. 4 shows the influenza A (H3N2) amplicons from real-time RRT-PCR reactions in FIG. 3 visualized on a 2% agarose gel.

FIG. 5 shows the real-time RT-PCR of influenza A (H3N2) RNA extracted from gelatin solutions.

FIG. 6 shows the cycle thresholds of Influenza A (H3N2) extracted from bovine gelatin.

### DESCRIPTION OF THE INVENTION

Standardized procedures for real-time (R) reverse transcription polymerase chain reaction (RT-PCR) testing from respiratory samples typically involve collection in viral transport medium (VTM). For clinical diagnostic testing using RRT-PCR, the World Health Organization (WHO) recommends RRT-PCR analysis on clinical samples collected in Copan's Universal Transport

Medium. Transport media (referred to as Universal Transport Medium (UTM) or more commonly, a Viral Transport Medium (VTM), or collection, transport and storage medium (CTS). These mediums are referred to herein as UTM, VTW, CTS or simply transport medium. Transport medium contains reagent blends optimized for preserving and maintaining clinical sample viral viability for downstream culture. Many samples collected in commercial transport media are routinely subjected to RNA/DNA extraction and nucleic acid testing (NAT) such as real-time RT-PCR.

Transport medium is a complex mixture of buffer, reducing agent, stabilization agent, balanced salts, proteins, sugars, and antimicrobials. An important component of nearly all transport medium is the stabilizing agent that prevents breakdown of the nucleic acids and, basically, stabilizes the sample and components for later testing. Certain components present in most transport medium are inhibitory during nucleic acid isolation and/or testing (e.g., nucleic acid extraction) and subsequent RRT-PCR analysis and other NAT protocols. Consequently, stability must always be balanced against possible inhibitory aspects in testing. Existing commercial viral transport media (VTM), comprise reagent blends optimized for preserving and maintaining viral viability for downstream culture, but are routinely subjected to RNA/DNA extraction and nucleic acid testing (NAT) such as real-time RT-PCR. Significant inhibition of 2-5 cycle threshold (CT) values in real-time RT-PCR and PCR is observed when nucleic acids from influenza A virus(RNA virus), Mycobacterium tuberculosis (a DNA bacterium), and adenovirus Type 5 (DNA virus) are extracted and detected from commercial VTM compared to PrimeStore MTM and positive controls(nuclease-free water). Gelatin, a common reagent used in VTM recipes, inhibits RNA extraction and RT-PCR amplification in a concentration dependent fashion.

It was surprisingly discovered that certain gelatins not currently incorporated into VTM can function as stabilizing agents for transport medium and at concentrations that do not inhibit or interfere with downstream processing. Using transport media formulations with fish-derived or other low molecular weight gelatin, it was surprising discovered that necessary stability could be achieved without interfering with NAT procedures as compared with conventional transport medium. Furthermore, the source of gelatin, but not specifically Bloom value was found to affect the degree of inhibition. Accordingly, compositions of the invention are useful for the collection, transport and storage of biological and other samples and at the same time maximize subsequent analysis, whether that analysis is nucleic acid testing or another diagnostic procedure.

Gelatin is derived from collagen which is a group of naturally occurring proteins found in animals, especially in the flesh and connective tissues of vertebrates in which collagen is a principle component. Collagen comprises a heterogeneous mixture of water-soluble peptides and proteins of high average molecular masses that can be completely or partially hydrolyzed as desired. Hydrolyzing breaks down the natural molecular bonds between individual collagen strands allowing them to reform and rearrange more easily. Once hydrolyzed, the proteins and peptides are soluble in most any polar solvent forming a gelatin solution. Gelatin solutions melt to a liquid when heated and solidify when cooled again. Solution of gelatin have a high viscosity and show visco-elastic flow and streaming birefringence, forming semi-solid colloid compositions. Gelatins transition from solid to liquid over a small temperature range depending, of course on temperature, but also on gelatin grade and concentration.

Gelatins are commercially available from a wide variety of readily available animal sources including, preferably, avian, bovine, equine, leporine, murine, ovine, piscine (ichthyic), and porcine. Also preferably, gelatin is obtained or derived from fish and fish products. Gelatin is prepared from such animal sources by boiling or simply heating which removes and hydrolyzes collagen that is present in the tissues. Suitable collagen-containing tissues include, but are not limited to skin, tendons, ligaments, crushed bone, horn and hooves, connective tissues, and many organs and intestines. Type A gelatin is derived from acid-cured or treated tissue and Type B gelatin is derived from alkaline-cured or treated tissue. Anhydrous gelatin is colorless and fairly brittle with the most common form being irreversibly hydrolyzed animal collagen. Anhydrous gelatin is manufactured as a powder or pellets which can be formed into a gelatinous liquid upon mixing with water.

Within the certain industries, such as cosmetics and personal care, the term plant collagen has appeared. Plant collagens are vegetable substitutes for animal collagen, such as for example, hydrolyzed wheat proteins. Typically, hydrolyzed plant proteins are manufactured from insolubilized flour proteins or gluten. These protein sources comprise a mixture of soluble and insoluble materials which can be hydrolyzed or otherwise processed (e.g. by fermentation) to become water soluble. Plant collagen is made by modifying polysaccharides through fermentation of yeast followed by heating and often denaturation to form gelatins. These plant-derived gelatins are biologically inactive and function well as stabilizing agents.

Gelatins are inexpensive and widely available commercially. Most gelatins are easily processed and purified to a desired specification including, for example, to a desired molecular weight range, to most any desired degree of hydrolysis, and/or under acid (Type A) or alkaline (Type B) conditions. The mechanical property of a gelatin such as gel strength is quantified using the Bloom test. As such, the mechanical strength is dependent on temperature variations, previous thermal history of the gel, and time. The Bloom test determines the weight (in grams) needed by a probe to deflect the surface of a gel solution a set distance without breakage. Using a one half inch probe, that distance is 4 mm. The result is expressed in Bloom grades which are most typically expressed as between about 30 and 300 and proportional to average molecular mass. A Bloom number of 50 to 125 is considered low with the composition having an average molecular mass of 20,000 to 25,000. A Bloom number of 175 to 225 is considered medium with the composition containing an average molecular mass of 40,000 to 50,000. A Bloom number of 225 to 325 is considered high with the composition containing an average molecular mass of 50,000 to 100,000.

### Exemplary Formulations

Conventional media and formulations can include, but is not limited to, collection and transport media that are currently approved by the Centers for Disease Control (CDC) for the collection and transport of biological samples and typically contains interfering substances that inhibit or interfere with the processing of the microorganisms suspected of being present in a sample, or any population of polynucleotides contained therein, such as, but not limited to, nucleic acid extraction, amplification, sequencing and characterization.

Transport medium formulations of the invention contain a stabilizing agent that does not interfere with NAT and other molecular analyses. The interfering substances may be absent from the stabilizing agent, separated from the stabilizing agent during processing, or removed as necessary by molecular techniques such as, for example, dialysis, salt or acid extraction, chromatography techniques, or other methods well know in the art. One preferred embodiment of the invention is directed to a transport medium containing gelatin, and preferable a gelatin obtained or derived from fish or fish products. Transport medium containing fish gelatin is generally of low average molecular mass having a low Bloom number grade. Transport medium containing fish gelatin instead of porcine or bovine are therefore particularly well suited for collecting, storing and transporting microorganisms, such as bacteria, fungi and viruses, obtained from a sample. In some embodiments the collection and transport medium is compatible with downstream processing and analyzing of pathogens, preferably human pathogens. In particular embodiments, the collection and transport medium is able to collect, store and/or transport samples containing, for example, Chlamydia, Mycoplasma, Ureaplasma, or viruses such as Adenovirus, Influenzavirus or RSV, or any combination thereof, including without limitation, to predict and help manage shift and drift and to manage an imminent or ongoing pandemic. In some embodiments, the collecting and transporting medium is capable of maintaining the viability of the microorganisms contained therein until the microorganism of interest is able to be cultured.

In certain embodiments, the collection, transport or storage medium is compatible with the isolation or purification of one or more nucleic acids from the biological sample and the performance of at least a first thermal cycling reaction on at a least a first nucleic acid so isolated or purified. A thermal cycling reaction can include, without limitation, PCR-based methodologies, as well as the addition of thermal cycling reaction reagents, heating or cooling phases, the amplification of a population of polynucleotides, the maintenance of a particular temperature, and the collection of a thermal cycling or amplification product. For example, a significant reduction (3-4 CT, or 10-fold differences) in cycle threshold (CT) values during RRT-PCR was observed when equal amounts of whole influenza A (H3N2) virus were extracted from commercial VTM compared to both PrimeStore Molecular Transport Medium and nuclease-free water control. Inhibition during RNA extraction and RT-PCR amplification could arise from gelatin present in collection media used for transport of the clinical sample. Thus, a matrix of gelatin is inhibitory to viral RNA extraction and RT-PCR amplification in a concentration dependent fashion. Furthermore, the source of gelatin also affects the level of inhibition.

The collection and transport solution of the present invention provides a number of improvements and benefits over those presently available in the art. Exemplary benefits include, without limitation, one or more of the following: compatibility with a variety of conventional nucleic acid extraction, purification, and amplification systems, genomic or meta-genomic analysis (e.g., sequencing), and any other suitable methods and techniques; compatibility with conventional microbial culturing techniques for propagation purposes; preservation of nucleic acid integrity within the sample; maintenance of high-quality, high-fidelity populations of nucleic acids during downstream molecular or chemical detection, analysis, or characterization of the medium containing the biological sample; facilitation of transport and shipping of the medium contacted with the biological sample at ambient temperatures, even over extended periods of time, or extreme temperature variations; suitability for short- (several hours to several days), intermediate- (days to several weeks), or long- (weeks to several months) term storage of the isolated nucleic acids.

In one aspect of the invention, the present invention provides for a medium that, when contacted with a sample, enables the rapid detection of a particular polynucleotide sequence. In an overall and general sense, the medium contacted with the sample allows for amplification of a population of polynucleotides suspected of containing the particular sequence of interest using conventional methods such as PCR and forward and reverse primers that are specific for the target sequence, hybridization of a specific probe set with the resulting PCR product, and performing analysis such as melting curve analysis. The present invention also concerns nucleic acid compositions, including, without limitation, DNA, RNA and PNA, isolatable from one or more biological samples or specimens using the collection, storage and transport medium of the invention.

In some embodiments of the compositions and methods of the present invention, the molecular or chemical detection, analysis, or characterization of the sample contacted with the CTS medium of the present invention is not substantially interfered with or inhibited by interfering substances contained in the CTS medium. In some embodiments, when the sample contacted with the CTS medium of the present invention is processed, there is at least an about 10 percent improvement as compared to when similar or the same type of samples contacted with conventional media are processed. In other embodiments there is at least about an 8 percent improvement, at least about a 6 percent improvement, and in some instances at least about a 5 percent, 4 percent, 3 percent, 2 percent or 1 percent improvement over when conventional medium is used.

### Biological Specimen Collection and Handling

Collection of a biological sample or specimen is a first step in many diagnostic platforms, propagation techniques, and molecular protocols requiring the isolation, detection and analysis of potentially minute amounts of nucleic acids from microorganisms including, but not limited to, bacteria, fungi and viruses. To facilitate the application of microbial detection and diagnostic strategies and their integration into the mainstream diagnostic laboratories there is a need for reliable, robust, and standardized collection systems developed specifically with the intent of being utilized for downstream processing such as nucleic acid based detection and testing, propagation of viral or microbial specimens in culture or both. The present invention affords such improvements through the use of new CTS media and formulations that display significant advantages over many of the commercially-available microorganism transport media. In addition, other solutions and mixtures (such as master mix for PCR) can also use noninterfering fish gelatin to stabilize proteins and other molecules.

The field of clinical molecular diagnostics changed drastically with the advent of polymerase chain reaction (PCR) in the mid eighties, and shortly thereafter with real-time PCR in the mid 90's. Real-time PCR (and RT-PCR) and real-time reverse transcriptase PCR (rRT-PCR) can deliver results in hours. Advances in other nucleic acid detection strategies (in addition to real-time PCR) such as transcription-mediated amplification, ligase chain reaction (LCR), microarrays, and pathogen gene chips and sequencing, have also contributed to a transition to the use of nucleic acid testing (NAT) in the clinical laboratory.

### Nucleic Acid Analyses

Nucleic acids obtained from biological samples collected, stored, or transported in one of the compositions of the invention are advantageously compatible with a number of conventional molecular and diagnostic isolation, purification, detection, and/or analytic methodologies. The compositions of the invention facilitate recovery, storage, and transport of populations of stabilized, substantially non-degraded, polynucleotides for use in a variety of downstream analyses including, without limitation, nucleic acid isolation, purification, amplification, and molecular analytical and/or diagnostic testing, assay, analysis, or characterization, and the like.

In certain embodiments, the nucleic acid(s) isolated by the methods of the present invention may serve as a template in one or more subsequent molecular biological applications, assays, or techniques, including, without limitation, genetic fingerprinting; amplified fragment length polymorphism (AFLP); restriction fragment length polymorphism analysis (RFLP); allele-specific oligonucleotide analysis (ASOA); microsatellite analysis; Southern hybridization; Northern hybridization; variable number of tandem repeats PCR (VNTR-PCR); dot-blot hybridization; PCR; quantitative real-time PCR; polymerase cycling assembly (PCA); nested PCR; quantitative PCR (Q-PCR); asymmetric PCR; DNA footprinting; single nucleotide polymorphism (SNP) genotyping; reverse transcription PCR (RT-PCR); multiplex PCR (m-PCR); multiplex ligation-dependent probe amplification (MLPA); ligation-mediated PCR (LmPCR); methylation specific PCR (MPCR); helicase-dependent amplification (HDA); overlap-extension PCR (OE-PCR); whole-genome amplification (WGA); plasmid isolation; allelic amplification; site-directed mutagenesis; high-throughput genetic screening; or the like, or any combination thereof.

A number of template dependent processes are available to amplify the marker sequences present in a given template sample. One of the best-known amplification methods is the polymerase chain reaction (referred to as PCR) which is described in detail e.g., in U.S. Pat. Nos. 4,683,195, 4,683,202 and 4,800,159 (each of which is specifically incorporated herein in its entirety by express reference thereto. Another method for amplification is the ligase chain reaction (“LCR”), disclosed, e.g., in EPA No. 320 308, and U.S. Pat. No. 4,883,750, each of which is incorporated herein in its entirety by express reference thereto. An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention.

Suitable standard hybridization conditions for the present invention include, for example, hybridization in 50% formamide, 5× Denhardts' solution, 5× SSC, 25 mM sodium phosphate, 0.1% SDS and 100 μg/ml of denatured salmon sperm DNA at 42° C. for 16 h followed by 1 hr sequential washes with 0.1×SSC, 0.1% SDS solution at 60° C. to remove the desired amount of background signal. Lower stringency hybridization conditions for the present invention include, for example, hybridization in 35% formamide, 5× Denhardts' solution, 5×SSC, 25 mM sodium phosphate, 0.1% SDS and 100 μg/ml denatured salmon sperm DNA or E. coli DNA at 42° C. for 16 h followed by sequential washes with 0.8×SSC, 0.1% SDS at 55° C. Those of ordinary skill in the art will recognize that conditions can be readily adjusted to obtain the desired stringency.

### Biological Samples and Specimens

As used herein, a “sample” can include a portion of material, preferably biological material, containing or presumed to contain one or more microorganisms of interest. It thus may be a composition of matter containing nucleic acid, protein, or another biomolecule of interest. The term “template” typically refers herein to a DNA or RNA molecular sequence that is detected by NAT.

The term “sample” can thus encompass a solution, cell, tissue, or population of one of more of the same that includes a population of nucleic acids (genomic DNA, cDNA, RNA, protein, other cellular molecules, etc.). The terms “nucleic acid source,” “sample,” and “specimen” are used interchangeably herein in a broad sense, and are intended to encompass a variety of biological sources that contain nucleic acids, protein, one or more other biomolecules of interest, or any combination thereof.

Samples in the practice of the invention can be obtained fresh, or can be obtained after being stored for a period of time, and may include, for example, material(s) of a clinical, veterinary, environmental or forensic origin, or may be isolated from one or more sources, such as without limitation, foods and foodstuffs, beverages, and beverage ingredients, animal feed and commercial feedstocks, potable waters, wastewater streams, runoff, industrial wastes or effluents, natural water sources, groundwater, soils, airborne sources, or from pandemic or epidemic populations, epidemiological samples, research materials, pathology specimens, suspected bioterrorism agents, crime scene evidence, and the like.

Exemplary biological samples include, but are not limited to, whole blood, plasma, serum, sputum, urine, stool, white blood cells, red blood cells, buffy coat, swabs (including, without limitation, buccal swabs, throat swabs, vaginal swabs, urethral swabs, cervical swabs, rectal swabs, lesion swabs, abscess swabs, nasopharyngeal swabs, and the like), urine, stool, sputum, tears, mucus, saliva, semen, vaginal fluids, lymphatic fluid, amniotic fluid, spinal or cerebrospinal fluid, peritoneal effusions, pleural effusions, exudates, punctates, epithelial smears, biopsies, bone marrow samples, fluid from cysts or abscess contents, synovial fluid, vitreous or aqueous humor, eye washes or aspirates, pulmonary lavage or lung aspirates, and organs and tissues, including but not limited to, liver, spleen, kidney, lung, intestine, brain, heart, muscle, pancreas, and the like, and any combination thereof. In some embodiments, the sample may be, or be from, an organism that acts as a vector, such as a mosquito, or tick, or other insect(s).

### Sample Collection Systems and Diagnostic Kits

In the practice of the invention, the disclosed compositions may be used in a variety of sample collection systems. Exemplary such systems may incorporate one or more collection devices (e.g., a swab, curette, culture loop, etc.); and a collection vessel (e.g., a vial, ampule, flask, bottle, syringe, test tube, specimen cup, etc.) to contain one or more of the compositions disclosed herein, and subsequently store and/or transport the collected sample. Exemplary specimen collection devices include, without limitation, those described in one or more of U.S. Pat. Nos. 4,235,244; 4,707,450; 4,803,998; 5,091,316; 5,108,927; 5,163,441; 6,312,395; 7,311,671; 7,541,194; and 7,648,681 (each of which is specifically incorporated herein in its entirety by express reference thereto).

The collection vessel is preferably releasably openable, such that it can be opened to insert the one-step compositions and closed and packaged, opened to insert the sample and optionally a portion of the collection device and closed for storage and transport, or both. The collection vessel may use any suitable releasably openable mechanism, including without limitation a screw cap, snap top, press-and-turn top, or the like. Such systems may also further optionally include one or more additional reagents, storage devices, transport devices, and/or instructions for obtaining, collecting, transporting, or assaying one or more samples in such systems.

The following examples illustrate embodiments of the invention, but should not be viewed as limiting the scope of the invention.

### EXAMPLES

### Exemplary Preparations of PrimeMix™

1. One or more buffers at about 1 mM to about 1 M, e.g., Tris, citrate, MES, BES, Bis-Tris, HEPES, MOPS, Bicine, Tricine, ADA, ACES, PIPES, bicarbonate, phosphate.
2. One or more osmolarity agents at about 1 mM to about 1 M, e.g., cationic functionalized zwitterionic compounds, betaine, DMSO, foramide, glycerol, nonionic detergents, BSA, polyethylene, glycol, tetramethylammonium chloride.
3. One or more chelators, at about 0.01 mM to about 1 mM, e.g., EGTA, HEDTA, DTPA, NTA, EDTA, citrate anhydrous, sodium citrate, calcium citrate, ammonium citrate, ammonium bicitrate, citric acid, diammonium citrate, potassium citrate, magnesium citrate, ferric ammonium citrate, lithium citrate.
4. One or more dyes at about 0.01 mM to about 50 mM, e.g., fluorescein, 5-carboxy-X-rhodamine, ROX™.
5. One or more salts at about 50 mM to 1 M, e.g., potassium chloride, magnesium sulfate, potassium glutamate.
6. One or more polymerases at about 0.05 U to about 1 U, e.g., Taq, Pfu, KOD, Hot start polymerases, next gen. polymerases.
7. Deoxynucleoside triphosphates at about 0.1 mM to about 1 mM, e.g., dATP, dTTP, dGTP, dCTP, dUTP.

### Exemplary Preparations of PrimeStore™

1. A chaotrope at about 0.5 M to about 6 M, e.g., Guanidine thiocyanate, Guanidine hydrochloride, Guanidine isocyanate.
2. An anionic detergent at about 0.1% to about 1% (wt./vol.), e.g., lauroyl sarcosine (inter alia Na salt), Sodium dodecyl sulfate, Lithium dodecyl sulfate, Sodium glycocholate, Sodium deoxycholate, Sodium taurodeoxycholate, Sodium cholate.
3. A reducing agent at about 0.5 mM to about 0.3 M, e.g., TCEP, β-ME, DTT, formamide, or DMSO.
4. A chelator, at about 0.01 mM to about 1 M, e.g., Sodium citrate, EDTA, EGTA, HEDTA, DTPA, NTA, or APCA.
5. A buffer at about 1 mM to about 1 M, e.g., TRIS, HEPES, MOPS, MES, Bis-Tris, etc.)
6. An acid q.s. to adjust to a pH of about 6 to 7, preferably 6.4 to 6.8 (e.g., HCl or citric acid)
7. Nuclease-free water q.s. to desired final volume.

Optionally one or more of:

8. A surfactant/defoaming agent about 0.0001% to about 0.3% (wt./vol.), e.g., Antifoam A® or Tween®.
9. An alkanol at about 1% to about 25% (vol./vol.) (e.g., methanol, ethanol, propanol, etc.)
10. RNA or DNA about 1 pg to about 1 μg/mL.
Exemplary Preparations of Gelatin. Test solutions (1.0, 0.5, 0.25, 0.1 and 0.01%, 75 w/v) containing cell culture tested porcine, bovine and cold water fish gelatin 76 (Sigma Cat Nos. G1890, G9391, and G7041 respectively) were prepared using nuclease-free water. A positive control (gelatin-free) solution was also prepared using nuclease-free water.
RT-PCR using whole influenza A Virus. A total of 103 TCID50/mL whole influenza A (H3N2) virus (A/Texas/78209/2008) was added to each test solution. For the limit of detection study, a serial dilution of whole influenza A (H3N2) virus from TCID50/mL to TCID50/mL was added to each medium (BD Universal Transport Medium (UTM), or PrimeStore MTM) or nuclease-free water (Positive Control). Ribonucleic acid (RNA) was extracted using PrimeExtract (Longhorn Vaccines & Diagnostics) per manufacturer's instructions with elution performed using 30 μl pre-warmed (75° C.) nuclease-free water.

Real-time reverse transcription-polymerase chain reaction (RRT-PCR) was carried out using a 1× mastermix (PrimeMix™) containing real-time primers and probes specific for universal detection of influenza A RNA according to manufacturer's recommendations (Longhorn Vaccines & Diagnostics, San Antonio, Tex.). Amplification was performed using an ABI 7500 thermocycler (Life Technologies, Foster City, Calif.). RT-PCR thermocycling consisted of a RT incubation step at 50° C. for 20 minutes followed by hot start activation at 95° C. for 5 minutes and 40 amplification cycles at 95° C. for 15 seconds and 60° C. for 32 seconds, respectively.

VTM was originally developed for the purpose of osmo-regulating, preserving and stabilizing the integrity of phospholipid bilayers of collected clinical samples pending shipment/transport and subsequent culturing. VTMs used for transporting viruses, chlamydia, ureaplasmas and mycoplasmas are typically comprised of balanced salts, minimal nutrients and antimicrobials to inhibit growth of selected organisms. However, the use of commercial VTM for nucleic acid testing has become a typical part of routine surveillance and detection of pathogens from clinical samples.

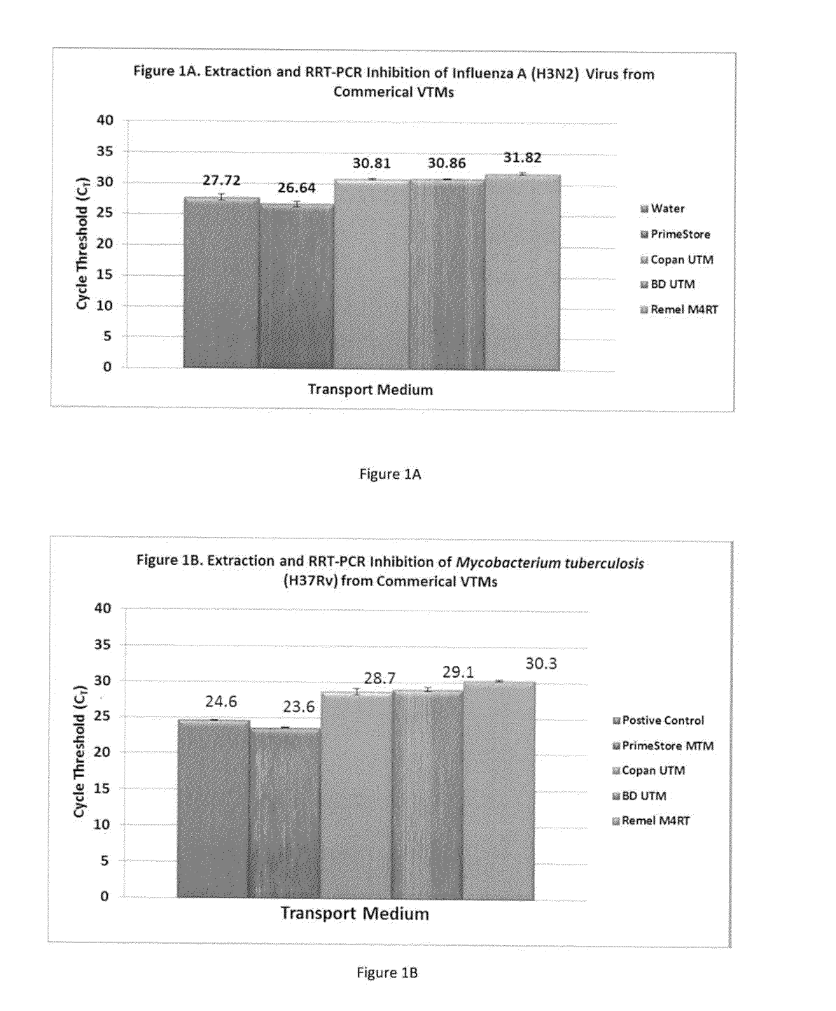

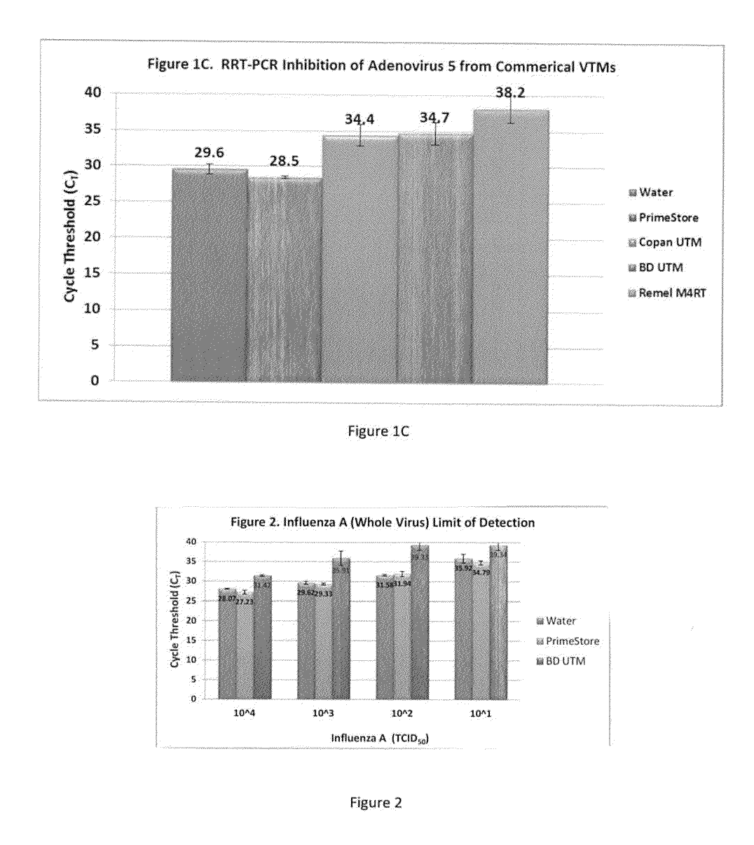

Equal amounts of whole influenza virus (TCID50/mL) was transferred into 3 commercial VTMs, PrimeStore MTM or nuclease-free water (positive control), and subsequently subjected to silica-based column extraction and real-time RT-PCR. Commercial VTM used were from Copan, Beckon Dickinson (BD) and REMEL. The formulation of these media are essentially identical and are those described by Racioppi et al., 1997 (6). The results for this experiment are shown in FIG. 1A. Three commercial VTMs exhibit an average 3-4 CT reduction in RRT-PCR compared to PrimeStore MTM and positive control (virus in nuclease-free water). Since 3.3 CTs equates to an average 10-fold differences in real-time PCR, an observed 3-4 CT difference in VTM compared to control represents a 10-fold reduction. Interestingly, PrimeStore MTM performed slightly better than positive water control reactions (FIG. 1A 120 and B).

Similarly, when CFU/mL of Mycobacterium tuberculosis (H37Rv; FIG. 1B), TCID50/mL of Adenovirus Type 5 (FIG. 1C) is spiked into each media, a significant difference in CT values (4-6 CT reduction) is observed in commercial VTM compared to PrimeStore MTM and water controls (FIG. 1B). This suggests that VTM inhibits DNA amplification during PCR. Since real-time PCR amplification of M. tuberculosis does not include a reverse transcription (RT) step, the observed CT reductions may be due to inhibition of Taq polymerase enzyme.

Serially diluted influenza A virus titration (TCID50/mL to TCID50/mL) reflected similar CT value differences corresponding to an average 10-fold reduction at each viral concentration tested using BD UTM compared to PrimeStore MTM and water control (FIG. 2). Importantly, at TCID50/mL and TCID50/mL viral concentrations, 3 of 4 replicate reactions were not detected by RT-PCR, compared to detection in all four replicates from PrimeStore MTM and positive control reactions.

Gelatin is typically present at a concentration of 0.5% in commercial VTM and, based on studies, is the most likely reagent responsible for VTM inhibition. Gelatin adds viscosity to the medium and is typically added to commercial VTM for stabilizing proteins from clinical samples for extended periods.

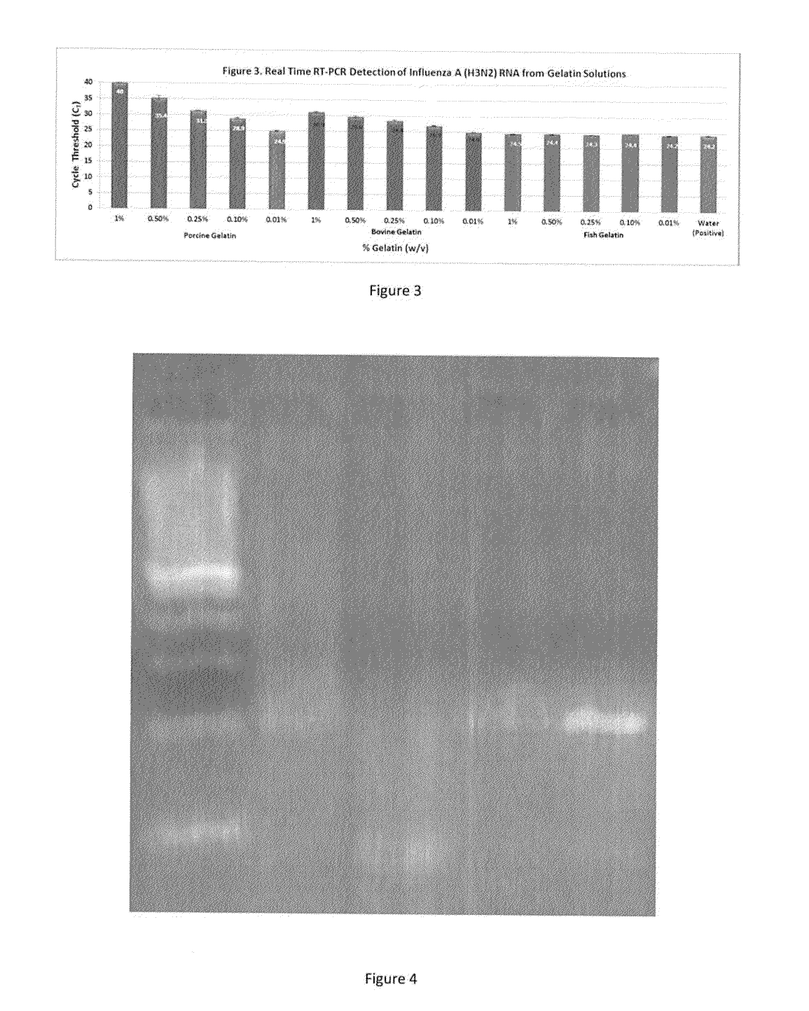

To demonstrate gelatin inhibition during real-time RT-PCR, solutions from gelatin bovine, porcine and cold water fish were prepared at 1.0, 0.5, 0.25, 0.1 and 152 0.01% (w/v) concentrations. To each gelatin dilution an equal amount of purified influenza viral RNA (viral copies) was added. Replicate extractions (4 reps) were performed for each gelatin dilution. The results indicate that Cold Water Fish gelatin performed similar to positive control reactions across all dilutions (FIG. 3). RRT-PCR inhibition of influenza A virus was noted in both bovine and porcine solutions, with no detection (i.e., CT=40) observed in 1% porcine gelatin (FIG. 3). This experiment demonstrates that gelatin (particularly at 1.0-0.1% concentrations) has a direct inhibitory effect on PCR ‘mastermix’ during real-time RT-PCR.

PCR products from post RRT-PCR reactions in FIG. 3 were further analyzed on a 2% agarose gel stained with ethidium bromide to assess whether gelatin inhibition was due to interference of PCR amplification directly, or simply fluorometer impediment during real-time fluorescence readings (FIG. 4). Lane 2, 3, and 4 depict amplification reactions from 1% fish, porcine, and bovine, respectively. The absence of an amplicon in lane 3 corresponds to the no amplification, i.e., CT=40 noted in 1% porcine (FIG. 3). Lane 1 and 5 is 100 by ladder and positive control, respectively. This indicates that the observed gelatin inhibition during real-time RT-PCR is not attributed to instrument readings by the fluorometer, but rather is due to gelatin directly inhibiting the molecular process of PCR.

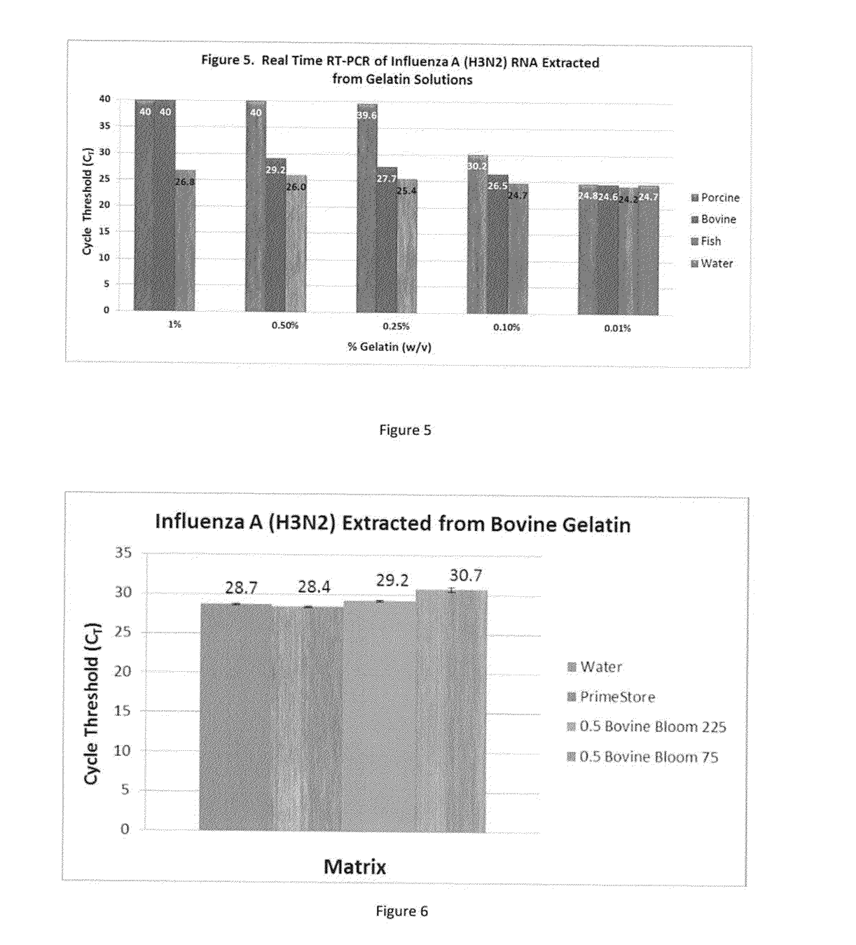

To determine whether gelatin also inhibits RNA extraction techniques, gelatin solutions from FIG. 3 containing spiked total influenza A RNA were subjected to RNA purification using traditional silica spin column extraction (FIG. 5). Replicate (N=4) extractions were performed for each gelatin solution and compared to equivalent amounts of influenza A RNA in nuclease-free water. Purifying naked RNA from gelatin matrices may improve the cycle threshold reductions noted during RRT-PCR amplification directly from gelatin dilutions containing spiked viral RNA. FIG. 4 reveals that RNA extraction did not improve the RRT-PCR inhibition noted in porcine and bovine gelatin dilutions at 1.0, 0.5 and 0.25% compared to water positive control. Real-time RT-PCR of viral RNA from cold water fish was similar to water controls at all dilutions.

These results indicate that porcine and bovine gelatin, at concentrations from 1.0 to 0.1%, are inhibitory to silica based extraction (FIG. 5). The molecular mechanism for this inhibition remains unknown. Proteins that comprise gelatin could be binding directly to RNA, or alternatively binding in a competitive fashion or via clogging to the silica dioxide in the columns. Whatever the molecular mechanism, the proteins that comprise gelatin appear to carry over into the reaction causing inhibition in PCR.

In another experiment, Bloom values (i.e., 225 and 75) of two bovine gelatins were evaluated to assess their effect on gelatin inhibition during RRT-PCR amplification of influenza A virus (FIG. 6). Gelatin strength is expressed in (gram) Bloom, where Bloom is the mass in grams necessary to produce the force which, applied to a plunger 12.7 mm in diameter, makes a depression 4 mm deep in a gel having a concentration of 6.67 per cent m/m and matured at 10° C. Commercial gelatins vary from low Bloom (<150), medium Bloom (150-220), to high Bloom (>220) types.

Equal amounts of TCID50/mL of whole influenza A virus were added to 0.5% solutions of bovine gelatin (Type B, cell culture tested) with Bloom values of 225 (classified as high bloom) and 75 (low Bloom), and compared to water control reactions (FIG. 6). Gelatin solutions with spiked influenza A virus were amplified using RRT-PCR and the CT values were determined.

The results indicate that bovine gelatin Bloom values do not affect the level of inhibition. Both 225 and 75 Bloom bovine gelatins, representing High and Low Bloom gelatin, respectively showed a similar reduction in CT (1 CT value) compared to positive water controls (FIG. 6). Gelatin is an amphoteric protein produced by partial hydrolysis of collagen fibers which contain a high concentration of glycine and proline. Addition of gelatin increases the medium viscosity and is purported to impart a cryoprotective effect on the collected sample. The majority of commercially available VTMs contain gelatin. Although these preparations were initially developed for the purpose of preserving the viability of viral pathogens for study, the use of VTM has expanded to include direct nucleic acid testing using extraction kits, PCR and sequencing applications. However, gelatin and the type of gelatin can negatively affect extraction and subsequent PCR amplification.

Collection and transport of biological specimens is a critical step in detection of pathogens using molecular diagnostics. PrimeStore Molecular Transport Medium (MTM) is a collection and transport solution optimized for NAT. PrimeStore MTM lyses phosopholipid bilayers and subsequently preserves and stabilizes released RNA/DNA for prolonged periods at ambient temperature. PrimeStore is designed to kill microbes in samples and therefore specimens preserved in PrimeStore MTM are not viable and cannot be cultured. However, the released RNA and DNA remain preserved and stabilized for extended periods at ambient temperature for downstream NAT.

In summary, commercial VTM inhibits real-time RT-PCR and are not ideal for molecular diagnostics. In this study commercial VTM and gelatin (a VTM component) inhibited real-time RT-PCR and were not improved by using a traditional silica based extraction method prior to amplification. Gelatin type and concentration in a medium directly affect the level of inhibition, whereas bloom values do not affect overall CT values during RRT-PCR.

Other embodiments and uses of the invention will be apparent to those skilled in the art from consideration of the specification and practice of the invention disclosed herein. All references cited herein, including all publications, all priority documents, all U.S. and foreign patents and patent applications identified herein, and U.S. Pat. No. 8,084,443 which issued Dec. 27, 2011, U.S. Pat. No. 8,080,645 which issued Dec. 20, 2011, U.S. Pat. No. 8,097,419 which issued Jan. 17, 2012, and International Application No. PCT/US2012/35253 filed Apr. 26, 2012, including the priority documents of each, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A method of detecting a sequence in a biological sample comprising:
contacting the biological sample at ambient temperature with an effective amount of a transport medium containing a stabilizing agent that functions to maintain the integrity of nucleic acid sequences contained within the biological sample without interfering with subsequent molecular analysis of the sample.

2. The method of claim 1, wherein the biological sample comprises cells suspected of being infected with a pathogen.

3. The method of claim 2, wherein the pathogen is a viral, a bacterial, a parasitic or a fungal infection.

4. The method of claim 1, wherein the transport medium is a collection, transport and storage medium and provided in at least a volume equivalent to the volume of the biological sample.

5. The method of claim 4, wherein the effective amount of CTS medium is a volume that at least three times the volume of the biological sample.

6. The method of claim 1, wherein the stabilizing agent is selected from the group consisting of a serum albumin, agar, carrageenan, gelatin, pectin, a sugar polymer, a galactose polymer, a polysaccharide, a heteropolysaccharide, a linear-sulfated polysaccharide, a protein, a collagen or a hydrolyzed product of collagen, and a combination thereof.

7. The method of claim 1, wherein the molecular analysis is PCR.

8. The method of claim 7, wherein the PCR analysis has a polymerase chain reaction cycle threshold (C_{T}) value that equates to about 10 to 100-fold lower than that obtained when the biological sample is maintain in a medium containing bovine serum albumin.

9. The method of claim 1, wherein 80 percent or more of the nucleic acid sequences present in the biological sample are detectable.

10. The method of claim 1, wherein the nucleic acid sequences present in the biological sample remain detectable for at least 30 days.

11. The method of claim 1, wherein ambient temperature is from about 10 C to about 35 C.

12. The method of claim 1, wherein stabilizing agent is a gelatin obtained or derived from fish or fish products.

13. A method of culturing a predetermined microorganism from a biological sample comprising:
contacting the biological sample suspected of containing the microorganism with an effective of an aqueous collection, transport or storage (CTS) medium to form a mixture, where the CTS medium contains from about 1 to 10 g/L of a buffer, from about 10 to 50 g/L of trehalose, from about 1 to 10 g/L of a carbohydrate comprising at least one of glucose, sucrose, mannose, altrose, allose, idose, talose, fructose, methyl-α-D-glucoside, galactose, ribose, deoxyribose, xylose, lactose, maltose, glycogen, amylase, cellulose, 6-deoxy-α-d-gluco-heptopyranosyl 6-deoxy-α-d-gluco-heptopyranoside, or (6-deoxy-α-d-gluco-heptopyranosyluronic acid) 6-deoxy-α-d-gluco-heptopyranosiduronic acid, or any combination thereof; and up to about 2 g/L of a serum albumin that does not interfere with quantitative detection of nucleic acid sequences by subsequent molecular analysis procedure;
inoculating a growth medium suitable for the microorganism with an effective amount of the mixture; and
culturing the mixture under conditions and for a period of time sufficient to obtain growth of the microorganism.

14. An aqueous collection, transport and storage (CTS) medium that comprises about 1 to about 10 g/L of a buffer, about 10 to about 50 g/L of trehalose; about 1 to about 10 g/L of a carbohydrate comprising glucose, sucrose, mannose, altrose, allose, idose, talose, fructose, methyl-α-D-glucoside, galactose, ribose, deoxyribose, xylose, lactose, maltose, glycogen, amylase, cellulose, 6-deoxy-α-d-gluco-heptopyranosyl 6-deoxy-α-d-gluco-heptopyranoside, or (6-deoxy-α-d-gluco-heptopyranosyluronic acid) 6-deoxy-α-d-gluco-heptopyranosiduronic acid, or any combination thereof; and up to about 2 g/L of a serum albumin, wherein the medium contains no substances at a concentration that interferes with the detection of nucleic acid as compared to a medium containing bovine gelatin.

15. The aqueous medium of claim 14, wherein the interfering substance is at least one of a sugar polymer, a galactose polymer, a polysaccharide, a linear sulfated polysaccharide, a heteropolysaccharide, collagen or a product of hydrolyzed collagen, a protein, or any combination thereof.

16. The aqueous medium of claim 14, further comprising about 30 to about 40 g/L of trehalose, about 5 to about 10 g/L of each of fructose and glucose; and from about 1 to about 1.5 g/L of a serum albumin.

17. The aqueous medium of claim 14, wherein the buffer comprises N-tris(hydroxymethyl)methyl-2-aminoethanesulfonic acid (TES), 4-(2-hydroxyethyl)-1-piperazineethanesulfonic acid (HEPES), 4-(2-hydroxyethyl)piperazine-1-propanesulfonic acid (HEPPS), 2-morpholinoethanesulfonic acid monohydrate (MES), 3-morpholinopropanesulfonic acid (MOPS), piperazine-N,N′-bis(2-ethane sulfonic acid) (PIPES), N-tris(hydroxymethyl)methyl glycine (Tricine), or a combination thereof.

18. The aqueous medium of claim 14, further comprising at least one antibiotic which comprises vancomycin, polymyxin B, gentamycin, colistin, trimethoprim, amphotericin B, or a combination thereof.

19. The aqueous medium of claim 14, further comprising a salt which comprises calcium chloride, potassium chloride, magnesium chloride, sodium chloride, magnesium sulfate, or a combination thereof.

20. The aqueous medium of claim 14, further comprising a pH indicator which comprises neutral red, phenol red, or a combination thereof.

21. The aqueous medium of claim 14, further comprising at least one amino acid which comprises glutamic acid, aspartic acid, or a combination thereof.

22. An aqueous collection, transport and storage (CTS) medium that comprises:
about 0.001 mM to about 650 mM of trehalose, about 0.001 mM to about 600 mM of a carbohydrate comprising allose, altrose, arabinose, deoxyribose, erythrose, erythrulose, fructose, galactose, glucose, idose, lyxose, mannose, psicose, ribose, ribulose, sorbose, tagatose, talose, threose, xylose, xylulose, cellobiose, isomaltose, lactose, lactulose, maltose, maltulose, mannobiose, melibiose, sucrose, turanose, xylobiose, 6-deoxy-α-d-gluco-heptopyranosyluronic acid, 6-deoxy-α-d-gluco-heptopyranosiduronic acid, 6-deoxy-α-d-gluco-heptopyranosylic acid, 6-deoxy-α-d-gluco-heptopyranoside, cellulose, dextran, galactan, glycogen, levan, maltodextrin, maltotriose, mannan, melezitose, methyl-α-D-glucoside, raffinose, rhamnose, starch, or a combination thereof;
about 1 μM to about 1 M of a buffer comprising BES, TES, HEPES, HEPPS, MES, MOPS, PIPES, Tricine, or a combination thereof;
up to about 2 g/L of a serum albumin;
about 1 μM to about 100 mM of a salt comprising calcium chloride, potassium chloride, magnesium chloride, sodium chloride, magnesium sulfate, or a combination thereof;
about 0.1 μM to about 500 mM of an amino acid comprising glutamic acid, aspartic acid, or a combination thereof;
about 1 μM to about 50 mM of a pH indicator comprising neutral red, phenol red, or a combination thereof; and
about 0.1 μM to about 1 mM of an antibiotic comprising vancomycin, polymyoxin B, gentamycin, colistin, trimethoprim, amphotericin B, or a combination thereof.

23. An aqueous collection, transport and storage (CTS) medium that comprises:
about 0.001 mM to about 650 mM of trehalose;
about 0.001 mM to about 600 mM of at least a second carbohydrate comprising allose, altrose, arabinose, deoxyribose, erythrose, erythrulose, fructose, galactose, glucose, idose, lyxose, mannose, psicose, ribose, ribulose, sorbose, tagatose, talose, threose, xylose, xylulose, cellobiose, isomaltose, lactose, lactulose, maltose, maltulose, mannobiose, melibiose, sucrose, turanose, xylobiose, 6-deoxy-α-d-gluco-heptopyranosyluronic acid, 6-deoxy-α-d-gluco-heptopyranosiduronic acid, 6-deoxy-α-d-gluco-heptopyranosylic acid, 6-deoxy-α-d-gluco-heptopyranoside, cellulose, dextran, galactan, glycogen, levan, maltodextrin, maltotriose, mannan, melezitose, methyl-α-D-glucoside, raffinose, rhamnose, starch, and any combination thereof;
about 0.001 mM to about 1000 mM of a buffer comprising BES, TES, HEPES, HEPPS, MES, MOPS, PIPES, Tricine, or any combination thereof; and
from about 1 to about 35 μM of a serum albumin.

24. The aqueous medium of claim 23, further comprising:
about 1 μM to about 100 mM of a salt comprising calcium chloride, potassium chloride, magnesium chloride, sodium chloride, magnesium sulfate, or any combination thereof;
about 0.1 μM to about 500 mM of an amino acid comprising glutamic acid, aspartic acid, or a combination thereof;
about 1 μM to about 50 mM of a pH indicator comprising neutral red, phenol red, or a combination thereof; or about 0.1 μM to about 1 mM of an antibiotic comprising vancomycin, polymyoxin B, gentamycin, colistin, trimethoprim, amphotericin B, or any combination thereof.

25. The aqueous medium of claim 23, which has a pH of about 7.1 to about 7.5.

26. The aqueous medium of claim 23, which is capable of sustaining the viability of a microorganisms in the biological sample, and contains no substances at concentrations that interfere with quantitative nucleic acid detection of a sequence of the microorganism by molecular analysis.

27. The aqueous medium of claim 26, wherein the biological sample containing the microorganisms is maintained in the CTS medium at a temperature of from about minus 80 C to about 37 C, for a period of from about one hour to about six months.

28. The aqueous medium of claim 26, wherein the biological sample containing the microorganism is maintained in the CTS medium at a temperature of from about OC to about 30 C, for a period of from about one to about 90 days.

29. The aqueous medium of claim 26, wherein the microorganism is Chlamydia, Mycoplasma, Ureaplasma, Adenovirus, Herpes Simplex Virus, Paramyxovirus, or Influenza virus.

30. The aqueous medium of claim 29, wherein the Influenza virus is one or more of Influenza A, Influenza B, or Influenza C, or Influenza H1, H3, H5, or H1N1 subtype.

31. The aqueous medium of claim 29, which is compatible with isolation, detection, purification, and amplification of a nucleic acid sequence of the biological sample.

32. The aqueous medium of claim 29, wherein amplification of the nucleic acid sequences is greater than or equal to about 1-log to the base 10 (about 3.3 C_{T}) when the sample is contacted by a conventional medium including at least one interfering substance.

33. A sample collection system that comprises:
a sterile collection device adapted to obtain a biological sample suspected of containing a microorganism; and
a sterile specimen container comprising the aqueous medium of claim 29 to which the biological sample is to be added.

34. The system of claim 33, wherein the microorganism is one or more of Chlamydia, Mycoplasma, Ureaplasma, Adenovirus, Herpes Simplex Virus, Paramyxovirus, or Influenza virus.

35. A method for amplifying a sequence from a nucleic acid contained in a biological sample comprising:
contacting the biological sample with an effective amount of the aqueous medium of claim 28 to form a mixture;
storing the mixture for a period of 30 days or more at a temperature of from about minus 10 C to about 35 C such that such at least 80 percent of the nucleic acid of the biological sample remain detectable;
amplifying the sequence of the stored mixture; and
detecting the amplified sequence.

36. The method of claim 35, wherein the period of time is greater than 3 months.

37. The method of claim 35, wherein detecting the amplified sequence is determined by measuring the polymerase chain reaction cycle threshold (C_{T}) of the sequence.

38. The method of claim 35, wherein the polymerase chain reaction cycle threshold (C_{T}) value equates to about 10 to 100-fold lower than that obtained when a substantial amount of an interfering substance is present in the composition.
